(19) United States
(12) Patent Application Publication
(10) Pub. No.: US 2022/0041881 A1
(43) Pub. Date: Feb. 10, 2022

(54) CARBOGENIC NANOPARTICLE-CONDUCTING POLYMER MATERIALS AND INKS FOR VOC AND MOISTURE SENSING, AND METHODS OF MAKING AND USING THE SAME

(71) Applicant: Novatec, Inc., Baltimore, MD (US)

(72) Inventors: Lopamudra BHATTACHARJEE, Kolkata (IN); Akshay SHARMA, Jhiwana (IN); Utpal MANNA, Kolkata (IN); Ayush GOEL, Karnal (IN); Biplab PAL, Ellicott City, MD (US); Conrad BESSEMER, Millersville, MD (US)

(73) Assignee: Novatec, Inc., Baltimore, MD (US)

(21) Appl. No.: 17/393,648

(22) Filed: Aug. 4, 2021

Related U.S. Application Data
(60) Provisional application No. 63/060,732, filed on Aug. 4, 2020.

(51) Int. Cl. C09D 11/52; C09D 11/037; C09D 11/033; C09D 11/108; H05K 1/09; H01B 1/24; B05D 1/00; B05D 3/06; B05D 7/00; G01N 27/12; G01N 33/00

(52) U.S. Cl. CPC C09D 11/52; C09D 11/037; C09D 11/033; C09D 11/108; H05K 1/097; H05K 2201/10151; B05D 1/005; B05D 3/062; B05D 7/54; G01N 27/127; G01N 33/0047; H01B 1/24

(57) ABSTRACT

The present disclosure is directed to a carbogenic nanoparticle polymer inks including a conducting polymer, such as those made of CQD-PPy and/or R-GO-PPy, methods of making the inks, and moisture and VOC sensors made therefrom.

### CROSS-REFERENCE TO RELATED APPLICATION

This application claims priority benefit under 35 U.S.C. § 119(e) to U.S. Provisional Application Ser. No. 63/060,732, filed Aug. 4, 2020, the contents of which is incorporated by reference herein in its entirety.

### FIELD OF THE DISCLOSURE

This disclosure is directed to carbogenic nanoparticle-based conducting polymer nanomaterial composite as colloidal suspension and as an ink, both of which are dispersed in water. The nanoparticle composite may be converted into a stable thin film by spin-coating and used in the sensing and detection of moisture and VOCs under average temperature, pressure, and humidity conditions.

### BACKGROUND OF THE DISCLOSURE

Chemi-resistive sensors are of industrial importance due to their simple structure, facile measurement, and cheap build-up. Chemi-resistive vapor sensors can detect volatile organic compounds (VOCs). Polar VOCs are alcohols, carbonyl compounds like aldehydes and ketones. Non-polar VOCs are of two types, aliphatic hydrocarbons, and aromatic hydrocarbons.

VOCs are associated with processes include rotting of fruit, bacterial decomposition, living cell degradation, virus replication cycle, etc., but also with materials, perfumes, odors, fruit smell, drying agents, paints, etc. Trace amounts of VOCs are produced from bacterial and virus infections within the human body. In those cases, the emitted particular VOCs become biomarkers for a particular disease. Hence early detection, monitoring, and determination in various stages of the disease can be done easily and efficiently by detecting specific VOCs or their mixtures in the exhaled breath of human beings, for example, for an early cancer diagnosis. Discerning between cancer patients and healthy subjects can be done by comparing their exhaled breath VOC profile via pattern recognition algorithms.

Portable and low-cost sensors for the ambient air monitoring of benzene and other volatile organic compounds have been developed for highly sensitive and real-time analysis of main compounds of interest consisting of aromatics such as benzene, toluene, xylene, and ethylbenzene (together they are industrially known as BTEX). Not all systems are able to detect a low ppb range of VOCs or a mixture of VOCs present in the breadth of patients, as the concentrations of VOCs in human breath are at low ppb levels.

The existing technology of VOC sensors has allowed the introduction of various types of low-cost sensors for air pollution monitoring, such as metal oxide sensors (MOx), amperometric or potentiometric electrochemical cells, photo-ionization detectors (PID), portable and micro-GC. Metal oxides with different shapes and architecture have been used to selectively detect VOCs.

U.S. Pat. No. 6,993,955 B1 and U.S. Pat. No. 6,994,777 B2 detail the utilization of conducting polymers as VOC sensor material as an alternative to metal oxide-based sensors. The sensor comprises at least one electrode pair and a photopolymerized electrically conducting polymer composition deposited in contact between each electrode pair. Each polymer composition may include an organic polymer capable of interacting with one or more analytes. The sensor provides the mean processing of the resultant electronic signal from each polymer composition and electrode pair.

Gas sensors based on conducting polymers fabricated using conducting polymers such as polyaniline (PAni), polypyrrole (PPy), and poly(3,4-ethylene dioxythiophene) (PEDOT) as the active layers have been reviewed. The conducting polymers are used as sensing materials by converting their thin films into transistors, optical sensors, and piezoelectric crystal sensors.

Some of the disadvantages associated with conducting polymer-based VOC sensor devices include: instability (which is the main drawback; the performances of this kind of sensor decreases dramatically over time due to the de-doping of conducting polymers when exposed to air); low selectivity between different VOCs, and the presence of other gases; and sensitive to moisture, so humidity must be considered when detecting other VOCs.

The sensing material is a key component in chemical sensors. Conducting polymers face specific issues that might limit the applications of this material as an active sensor component in detecting VOCs, especially those related to disease diagnostics. To increase the surface to volume ratio, selectivity and create more sites for VOC adsorption, nanomaterials have been incorporated as composite material along with conducting polymers.

As discussed in U.S. Pat. No. 8,366,630 B2, U.S. Pat. No. 8,683,672 B2, U.S. Ser. No. 10/697,918 B2, and US 2005/0000830 A1, carbon nanotubes (CNTs) have been used in conjugation with conducting polymers for VOC sensor applications. CNTs show excellent mechanical and electronic properties. However, CNTs show weak responses and low selectivity toward specific gas molecules due to the weak interaction between CNTs and analyte molecules.

U.S. Pat. No. 8,366,630 B2 provides a system for measuring biomarker analytes indicative of various diseases comprising an array of sensors sensitive to volatile organic compounds. Notably, the system is composed of a random network of single-walled carbon nanotubes (SWCNTs) coated with non-polar small organic molecules in conjunction with learning and pattern recognition algorithms.

US 2005/0000830 A1 is directed to sensor devices and methods that utilize carbon nanotubes as a chemically sensitive element. US 2012/0245854 A1 provides a system and method for diagnosing, monitoring, or prognosing Alzheimer's disease using at least one sensor comprising carbon nanotubes coated with cyclodextrin or derivatives thereof and/or at least one sensor comprising metal nanoparticles coated with various organic coatings in conjunction with a learning and pattern recognition algorithm.

US 2012/0245434 A1 provides a system and method for diagnosing, monitoring, or staging Parkinson's disease using at least one sensor comprising carbon nanotubes coated with cyclodextrin or derivatives thereof or metal nanoparticles coated with various organic coatings in conjunction with a learning and pattern recognition algorithm. The VOC sensing device (nano-sensor) includes a substrate with at least a pair of conductive electrodes spaced apart by a gap and an electrochemically functionalized semiconductive nanomaterial bridging the electrodes' gap from a nanostructure network.

A few other VOC sensors have been developed that include carbogenic nanomaterials in a supporting role and other nanomaterials. See PCT/KR2018/000733, U.S. patent application Ser. No. 14/840,694, and Journal of Polymer Materials 18(3):225-258 (teaching that colloidal nanoparticles in conjugation with conducting polymers have been used to stabilize the polymer into a processible colloidal material; and incorporating C60 (an n-type dopant) in PPy can result in a better conducting material with improved polarization properties).

A colloidal suspension of polypyrrole and CQD has been fabricated. Bhattacharjee, L., et al., “Stable Semiconducting Ink Based on a Polypyrrole/Carbon-Quantum-Dot Aqueous Colloidal Suspension: A Potential Sensor for Volatile Organics Present in Food,” ChemistrySelect, Vol. 2, Issue 6, pp. 2139-2143 (2017). Formation of CQD-PPy composite for detection of picric acid in water and soil is taught. Pal, A., et al., “Conducting Carbon Dot-Polypyrrole Nanocomposite for Sensitive Detection of Picric acid,” ACS Appl. Mater. Interfaces, Vol. 8, Issue 9, pp. 5758-5762 (2016). The process restricts the polymer's formation on the CQD surface, and results in unstable colloids not suitable for further processing like spin-coating.

The challenges in building a device that can accurately detect moisture and VOCs include: fabrication of a PCB using a colloidal ink; building a low-cost and portable device; and designing a sensor that can be regenerated and reused multiple times; and, for VOC detection, reducing the false-positive result due to hindrance of moisture.

### SUMMARY OF THE DISCLOSURE

An embodiment is a carbogenic nanoparticle-conducting polymer composite ink dispersed in water, wherein the carbogenic nanoparticle is reduced graphene oxide (R-GO). The carbogenic nanoparticle-conducting polymer composite ink may be selected from the group consisting of: R-GO-PPy composite ink, R-GO-PANI composite ink, R-GO-PTH composite ink, R-GO-PA composite ink, R-GO-PPP composite ink, R-GO-PPV composite ink, R-GO-PF composite ink, or a combination thereof. The composite ink may be R-GO-PPy composite ink, optionally having a viscosity of about 20 mPa·s to about 26 mPa·s within a temperature range between about 25° C. and about 50° C., and/or a zeta potential of about −3 mV to about −8 mV and a hydrodynamic radius from about 900 to about 2000 nm.

In another embodiment, a carbogenic nanoparticle-conducting polymer composite ink dispersed in water selected from the group consisting of: R-GO-PPy composite ink, CQD-PANI composite ink, R-GO-PANI composite ink, CQD-PTH composite ink, R-GO-PTH composite ink, CQD-PA composite ink, R-GO-PA composite ink, CQD-PPP composite ink, R-GO-PPP composite ink, CQD-PPV composite ink, R-GO-PPV composite ink, CQD-PF composite ink, R-GO-PF composite ink, or a combination thereof. The composite ink may have a viscosity of about 20 mPa·s to about 0.15 Pa·s within a temperature range between about 25° C. and about 50° C., and/or a zeta potential of about +40 mV to about −40 mV and having a hydrodynamic radius from about 40 to about 2000 nm.

Another embodiment includes a thin film coated PCB comprising a thin film of the carbogenic nanoparticle-conducting polymer composite ink, optionally wherein the thin film has a thickness of about 10 nm to about 50 nm, or about 15 nm to about 40 nm. The thin film may include CQD-PPy composite ink or R-GO-PPy composite ink.

Yet another embodiment is a method of making a thin film coated PCB comprising the steps of: treating the PCB under a UV lamp at about 260 nm to about 400 nm for about 15 minutes to about 60 minutes; and spin-coating the treated PCB with a carbogenic nanoparticle-conducting polymer composite ink including the steps of: i) horizontally positioning the treated PCB on a rotating disk of a spin-coating machine, under vacuum; ii) coating the substrate with a small amount of the composite ink; iii) rotating the coated PCB at one or more different speeds to obtain a first layer of composite ink on the PCB; and iv) optionally repeating steps ii and iii one to four times to obtain a double, triple, quadruple or quintuple layer of composite ink to make the thin film on the PCB. The method may also include step v) maintaining the coated PCB under vacuum for at least about 2 hours. Rotating the PCB at one or more different speeds may include three steps with each step being at a different rotation speed for about 20 seconds to about 60 seconds. Rotating the PCB at one or more different speeds may include (a) rotating the coated PCB for about 20 seconds to about 60 seconds at a speed of about 1000 RMP to about 1700 RPM, followed by (b) rotating the coated PCB for about 20 seconds to about 60 seconds at a speed of about 2000 RMP to about 3000 RPM, followed by (c) rotating the coated PCB for about 20 seconds to about 60 seconds at a speed of about 4000 RMP to about 6000 RPM. The thin film of composite ink may have a thickness of about 15 nm to about 40 nm. The thin film may be processed from: CQD-PPy composite ink, R-GO-PPy composite ink, CQD-PANI composite ink, R-GO-PANI composite ink, CQD-PTH composite ink, R-GO-PTH composite ink, CQD-PA composite ink, R-GO-PA composite ink, CQD-PPP composite ink, R-GO-PPP composite ink, CQD-PPV composite ink, R-GO-PPV composite ink, CQD-PF composite ink, R-GO-PF composite ink, or a combination thereof. The thin film may be processed from: CQD-PPy composite ink or R-GO-PPy composite ink.

Other embodiments include a VOC sensor or a moisture sensor comprising the thin film coated PCB. A method of detecting moisture, VOCs or both in a sample using the thin film coated PCB is also disclosed.

### BRIEF DESCRIPTION OF THE DRAWINGS

FIG. 1 depicts an exemplary detachable probe sensor with circular sensing component and detachable wire for connection to a Gateway box.

FIG. 2 depicts a Gateway box into which one or more moisture sensor probes can be connected via wire.

FIG. 3 depicts a schematic method of measuring the moisture in a polymer sample packet.

FIGS. 4A and 4B show the response with CQD-PPY composite ink and R-GO-PPy composite ink exposed to humidity.

FIGS. 5A and 5B are graphs of XPES data for CQD-PPY composite ink and R-GO-PPy composite ink.

FIG. 6 is a graph of VOC sensing data with CQD-PPY composite ink.

FIG. 7 is a graph of VOC sensing data with CQD-PPY composite ink exposed to ethanol.

FIG. 8 is a graph of VOC sensing data with CQD-PPY composite ink exposed to acetone.

FIG. 9 is a graph of VOC sensing data with CQD-PPY composite ink exposed to ethyl acrylate.

FIG. 10 is a graph of VOC sensing data with R-GO-PPY composite ink.

FIG. 11 is a graph of VOC sensing data with R-GO-PPY composite ink exposed to acetone.

FIG. 12 is a graph of VOC sensing data with R-GO-PPY composite ink exposed to ethanol.

### DETAILED DESCRIPTION OF THE DISCLOSURE

This disclosure relates to a novel synthesized highly absorbing and conducting carbogenic nanoparticle (CGNP)-conducting polymer composite material, which may optionally be reduced to a water-dispersible composite ink that acts as an active component for designing efficient sensors for moisture or VOCs.

CGNPs are an improved economical alternative to CNTs, for some or all of the following reasons: CGNPs can be synthesized readily via pyrrolytic methods, with considerable yields; CGNPs recently has gained popularity as a potentially sustainable alternative to CNTs; CGNPs are made solely out of carbon atoms; CGNPs are earth-abundant, and CGNPs are benign to environmental pollution or health issues. Examples of CGNPs used herein are carbon quantum dots (CQD) and reduced graphene oxide (R-GO).

To increase electron mobility and to make the flexible films of semiconducting polymers more conducting, the present disclosure incorporates either CQDs or R-GO with conducting polymers to make a composite ink. The electrical properties of conducting polymers are important, especially when these materials are used as chemi-resistive sensors. The electrical properties can be controlled by doping and undoping processes, resulting in conducting and nonconducting states. The electrical conductivity also depends on the type and amount of nanosized fillers used, producing the positive or negative carriers responsible for the conduction. Any interaction of these polymers that affects the number and movement of charge carriers affect the conductivity and is the main principle behind the VOC sensing characteristics. Advances in nanotechnology allow for the fabrication of various conducting polymer nanocomposites using different techniques. Conducting polymer nanocomposites have a high surface area, small dimension, and enhanced properties, making them suitable for various sensor devices.

This is the major advantage of the CGNPs, where surface-confined in situ polymerization of the monomer results in the formation of CGNP-conducting polymer composite, which can be stabilized as an ink.

Some of the advantages of the resultant composite ink includes: ability to work as a flexible substrate in moisture sensors (also referred to as humidity sensors) and VOC sensors; increased storage time at room temperature; processable by spin-coating on PCBs or other electrodes; improved dispersion (i.e., the CGNPs are well dispersed making the colloidal stability form better thin films).

Some advantages of certain embodiments of the disclosure include: one to one replacement of conventional conducting polymer-based sensors; usability without the need for approval of any governing body; environmentally friendly; inexpensive, easily scalable, and easy to use; easily stored; robust and inert to environmental fluctuation; operates faster compared to presently available sensors; may be regenerated at ambient conditions; able to distinguish between VOCs of the same homologous group; able to distinguish between VOCs of two different functional groups.

Chemi-resistive sensors for VOCs where carbon-based nanomaterials act as the active element is important for industrial applications due to the improvements, such as low-cost fabrication, miniaturization, and the capability of being integrated into a portable device for non-invasive disease diagnosis.

The disclosed technology introduces an affordable and portable technology that can solve issues in the art related to selectivity, type of response, effect of surrounding humidity and temperature, regeneration of the sensor, and detection of mixed VOCs. A sensor may work to detect to either detect moisture or selectively detect VOCs and their mixture in the presence of moisture.

A carbogenic nanoparticle (CGNP)-conducting polymer composite material (referred to herein as “the composite material”) disclosed herein comprises a carbogenic quantum dot coated with a conducting polymer, which may be processed as a colloidal suspension. Another carbogenic nanoparticle (CGNP)-conducting polymer composite material disclosed herein comprises reduced graphene oxide (R-GO) coated with a conducting polymer, which may be processed as a colloidal suspension. An advantage of these materials is that they may be processed as and reduced to a stable colloidal composite ink (also referred to as a “composite ink”), which is environmentally friendly and inexpensive without any added stabilizers.

Carbon quantum dots (CQDs) are zero-dimensional carbon-based nanomaterials known for their small size and relatively strong fluorescence characteristics. CQDs also exhibit good water solubility, chemical stability, and photobleaching resistance, ease of surface functionalization and large-scale preparation. large surface area, good conductivity, fast charge transfer of CQDs. Unique electronic and chemical structures of CQDs can be adjusted by their size, shape, surface functional groups, and heteroatom doping. Wang, X. et al., “A Mini Review on Carbon Quantum Dots: Preparation, Properties, and Electrocatalytic Application,” Front. Chem., Vol. 7, Art. 671, 9 pp. (2019). CQDs used herein may be small carbon nanoparticles less than about 10 nm in size.

Reduced graphene oxide (R-GO) is a form of graphene oxide (GO) that is processed by chemical, thermal and other methods in order to reduce the oxygen content, while GO is a material produced by oxidation of graphite which leads to increased interlayer spacing and functionalization of the basal planes of graphite. Reduced graphene oxide (R-GO) contains residual oxygen and other heteroatoms, as well as structural defects. Both GO and R-GO have an extremely high surface area.

Any conducting polymer known in the art may be used with the present disclosure. The conducting polymer may be polyaniline (PANT), polypyrrole (PPy), and poly(3,4-ethylene dioxythiophene) (PEDOT), polyacetylene (PA), polythiophene (PTH), poly(para-phenylene) (PPP), poly(phenylenevinylene) (PPV), and polyfuran (PF). The conducting polymer may be polyaniline (PANT), polypyrrole (PPy), polythiophene (PTH), or poly(3,4-ethylene dioxythiophene) (PEDOT). The conducting polymer may be polypyrrole (PPy).

In an embodiment, the carbogenic nanoparticle (CGNP)-conducting polymer composite material may be CQD-PPy composite material, R-GO-PPy composite material, CQD-PANI composite material, R-GO-PANI composite material, CQD-PTH composite material, R-GO-PTH composite material, CQD-PA composite material, R-GO-PA composite material, CQD-PPP composite material, R-GO-PPP composite material, CQD-PPV composite material, R-GO-PPV composite material, CQD-PF composite material, R-GO-PF composite material, or a combination thereof. The carbogenic nanoparticle (CGNP)-conducting polymer composite material may be R-GO-PPy composite material, R-GO-PANI composite material, R-GO-PTH composite material, R-GO-PA composite material, R-GO-PPP composite material, R-GO-PPV composite material, R-GO-PF composite material, or a combination thereof.

In certain embodiments, the composite ink formed from the carbogenic nanoparticle (CGNP)-conducting polymer composite material may be CQD-PPy composite ink, R-GO-PPy composite ink, CQD-PANI composite ink, R-GO-PANI composite ink, CQD-PTH composite ink, R-GO-PTH composite ink, CQD-PA composite ink, R-GO-PA composite ink, CQD-PPP composite ink, R-GO-PPP composite ink, CQD-PPV composite ink, R-GO-PPV composite ink, CQD-PF composite ink, R-GO-PF composite ink, or a combination thereof. The carbogenic nanoparticle (CGNP)-conducting polymer composite material may be R-GO-PPy composite material, R-GO-PANI composite material, R-GO-PTH composite material, R-GO-PA composite material, R-GO-PPP composite material, R-GO-PPV composite material, R-GO-PF composite material, or a combination thereof. The composite ink formed from the carbogenic nanoparticle (CGNP)-conducting polymer composite material may be R-GO-PPy composite ink, R-GO-PTH composite ink, R-GO-PANI composite ink, R-GO-PPP composite ink, R-GO-PPV composite ink, or a combination thereof.

The composite materials and inks disclosed herein are stable. Stable as used herein means that the composite material or composite ink may be stored at room temperature for over 10 days, about 10 to about 60 days, about 20 to about 45 days, about 30 days to about 45 days, or about 30 days without precipitation.

The carbogenic nanoparticle (CGNP)-conducting polymer composite material disclosed herein may be dispersed in water.

The synthesis of the carbogenic quantum dot may be achieved through in situ surface-confined oxidative polymerization or any other means known in the art.

The composite ink may be coated on any substrate, including metal, plastic, glass, and fabric. The composite ink may be spin-coated as a thin film on any printed circuit board (PCB). Any PCB known in the art may be used in connection with this disclosure. The thin film may be applied in one to five layers, or two to four layers. The thin film may be applied in 2 layers, 3 layers, or 4 layers. The thickness of the thin film on the PCB may be about 10 nm to about 50 nm, about 15 nm to about 40 nm, or about 20 nm to about 30 nm. The thickness of the thin film made from CQD-PPy composite ink may be about 15 nm to about 40 nm, about 20 nm to about 34 nm, about 25 nm to about 35 nm, or about 30 nm. The thickness of the thin film made from R-GO-PPy composite ink may be about 15 nm to about 40 nm, about 15 nm to about 30 nm, about 15 nm to about 25 nm, or about 20 nm.

A thin film of the carbogenic nanoparticle (CGNP)-conducting polymer composite ink may be applied to a PCB by spin-coating. When the carbogenic nanoparticle (CGNP)-conducting polymer composite material is dispersed in water, the process of spin-coating on a PCB, e.g., a copper surface, becomes increasing difficult. The inventors have developed a method of spin-coating of the composite ink disclosed therein to provide a thin film layer on a PCB which may be used as a sensing component in a VOC or moisture sensor.

For spin-coating, the composite ink may have specific properties, e.g., viscosity, ζ-potential, and hydrodynamic radius in order to provide a thin film with the desirable sensing properties. Colloidal particle diffusivities may be measured by light scattering and ζ-potentials determined from electrophoretic mobilities. A hydrodynamic size can be calculated from the diffusivity by use of the Stokes-Einstein equation, although this ignores the influence of the surface charge and the ion cloud surrounding each particle. Similarly, ζ-potentials are often calculated from a radius determined by transmission electron microscopy or light scattering. The ζ-potential is defined as the potential at the electrokinetic shear surface.

The viscosity (η) of the carbogenic nanoparticle (CGNP)-conducting polymer composite ink may be about 20 mPa·s to about 0.15 Pa·s, about 20 mPa·s to about 80 mPa·s, about 20 mPa·s to about 50 mPa·s, about 20 mPa·s to about 40 mPa·s, or about 20 mPa·s to about 30 mPa·s within a temperature range between about 25° C. to about 50° C. Zeta potential of the composite ink may be about +40 mV to about −40 mV, about +10 mV to about −30 mV, about +2 mV to about −20 mV, about −2 mV to about −15 mV, about −3 mV to about −12 mV, or about −4 mV to about −10 mV and having a hydrodynamic radius from about 40 to about 2000 nm, about 40 to about 200 nm, about 50 to about 150 nm, about 1000 to about 2000 nm, about 1100 to about 1700 nm, about 100 to about 500 nm, or about 500 to about 1000 nm.

The viscosity (η) of CQD-PPy composite ink may be about 20 mPa·s to about 30 mPa·s, about 24 mPa·s to about 30 mPa·s, about 25 mPa·s to about 27 mPa·s, about 26 mPa·s to about 27 mPa·s, or about 26 mPa·s to about 26.5 mPa·s within a temperature range between about 25° C. to about 50° C. Zeta potential of CQD-PPy composite ink is about −8 mV to about −12 mV, −9 mV to about −10 mV, or about −10 mV and having a hydrodynamic radius from about 40 nm to about 150 nm, or about 50 nm to about 120 nm.

The viscosity (η) of R-GO-PPy composite ink may be about 20 mPa·s to about 30 mPa·s, about 20 mPa·s to about 26 mPa·s, about 22 mPa·s to about 26 mPa·s, about 23 mPa·s to about 25 mPa·s, or about 23.5 mPa·s to about 24.6 mPa·s within a temperature range between about 25° C. to about 50° C. Zeta potential of R-GO-PPy composite ink is about −2 mV to about −8 mV, about −3 mV to about −6 mV, about −4 mV to about −5 mV, or about −4.38 mV and having a hydrodynamic radius from about 900 to about 2000 nm, or about 1100 nm to 1700 nm with signs of aggregations.

The process used to spin coat the composite ink on PCBs may be termed as Static Dispensing. In this process, the PCB may first undergo processing to make the surface hydrophilic. For example, the PCB may be pre-treated under a UV lamp at about 260 nm to about 400 nm, at about 300 to about 400 nm, or at about 340 to about 360 nm, for about 15 minutes to about 60 minutes, about 20 minutes to about 50 minutes, about 30 to about 40 minutes, or about 30 minutes to make the surface hydrophilic in order to have better adhesion of the composite ink on the metal of the PCB and adhesion of the PCB to the platform that holds the PCB. A thermosetting phenol formaldehyde resin formed from a condensation reaction of phenol with formaldehyde, such as Bakelite, may be used as the platform.

In the next step, the composite ink may be coated as a thin film on the PCB according to the following steps: i) horizontally positioning the PCB on a rotating disk of a spin-coating machine, under vacuum; ii) coating the substrate with a small amount of the composite ink; iii) rotating the coated PCB at one or more different speeds to obtain a first layer of composite ink on the PCB. Steps ii and iii may be repeated one to four times to obtain a double, triple, quadruple, or quintuple layer of composite ink to make the thin film on the PCB.

The spin-coated PCB may be maintained under vacuum at least about 2 hours before use in a sensor device. Any known spin-coating machine, such as a EZ-spin A1, Apex Instrument, and rotating disk known in the art may be used.

The step of rotating the PCB at one or more different speeds may include a number of steps with each step being a different rotation speed for the same or different amount of time. Each rotation step may be for about 20 seconds to about 60 seconds, about 20 seconds to about 40 seconds, or about 30 seconds.

Rotating the PCB at one or more different speeds may include: (a) rotating the coated PCB for 20 seconds to 60 seconds, about 20 seconds to about 40 seconds, or about 30 seconds at a speed of about 1000 RMP to about 1700 RPM, or about 1500 RPM, followed by (b) rotating the coated PCB for 20 seconds to 60 seconds, about 20 seconds to about 40 seconds, or about 30 seconds at a speed of about 2000 RMP to about 3000 RPM, or about 2500 RPM, followed by (c) rotating the coated PCB for 20 seconds to 60 seconds, about 20 seconds to about 40 seconds, or about 30 seconds at a speed of about 4000 RMP to about 6000 RPM, or about 5000 RPM. Rotating the PCB at one or more different speeds may include: (a) rotating the coated PCB for about 30 seconds at a speed of about 1500 RPM, followed by (b) rotating the coated PCB for about 30 seconds at a speed of about 2500 RPM, followed by (c) rotating the coated PCB for about 30 seconds at a speed of about 5000 RPM.

The small amount of composite ink may be adjusted based on the size of the electrode which will be envisioned by one of ordinary skill in the art. The small amount of composite ink may be about 0.1 ml to about 1 ml, about 0.25 ml to about 0.75 ml, about 0.2 ml to about 0.5 ml, about 0.5 ml to about 1 ml, or about 0.5 ml.

In an embodiment, CQD-PPy composite ink is applied by spin-coating on a PCB in accordance with the process set forth above.

In an embodiment, R-GO-PPy composite ink is applied by spin-coating on a PCB in accordance with the process set forth above.

The carbogenic nanoparticle (CGNP)-conducting polymer composite inks disclosed herein are temperature stable such that a sensing component made with the composite inks may be subjected to any conventional method for regeneration, such as blown with nitrogen or heated by a micro-heater to allow desaturation and removal of the VOC and/or moisture, and allow reuse of the sensing component.

The composite inks disclosed herein may be used as a sensing component in a moisture sensor or VOC sensor.

VOCs may be adsorbed by CGNP due to, e.g., their large specific surface area, rich porous structure, and high adsorption capacity. The CQD-PPy composite ink-coated substrate responds towards VOCs through adsorption.

CQD-PPy composite ink may be used to make a sensor that detects and differentiates between different VOCs for the following reasons: CQD-PPy composite ink may be spin-coated on the PCB to form a thin transparent film; CQD-PPy composite ink is not affected by or responsive to moisture, which reduces the false-negative results; and CQD-PPy composite ink is water-based and may be fabricated over the PCB, hence making it cost-effective. The CQD-PPy is used to provide a highly conductive ink that acts as the VOC sensor's sensing component.

An embodiment is a highly conductive ink comprising a carbogenic nanoparticle (CGNP)-conducting polymer composite material dispersed in water. A thin film comprising this highly conductive ink may act as the sensing component of a moisture sensor or a VOC sensor. Both the composite ink and the sensor (VOC or moisture) can be produced at relatively low expense using readily available, environmentally friendly materials.

In the VOC sensor, specific VOC compounds in the sample, such as air or breadth, may be identified using the specific patterns in current-voltage (I-V) obtained from the sensing component. In the moisture sensor, moisture in the sample, such as air or breadth, may be identified using the specific patterns in current-voltage (I-V) obtained from the sensing component.

The VOC sensor made with a sensing component including a thin film disclosed herein may detect very low concentration of (˜ppb) VOCs. The VOC sensor may detect VOCs rapidly, for example, in about 20 seconds to about 60 seconds, or within about 30 seconds of exposure. The VOC sensor may be regenerated (e.g., dried and ready for reuse) in about 50 seconds to about 150 seconds, in about 50 to about 100 seconds, or within about 100 seconds after each exposure.

The moisture sensor disclosed herein may detect moisture rapidly, for example, in about 20 seconds to about 60 seconds, or within about 30 seconds of exposure. The moisture sensor may be regenerated (e.g., dried and ready for reuse) in about 50 seconds to about 150 seconds, in about 50 to about 100 seconds, or within about 100 seconds after each exposure.

In an embodiment, R-GO-PPy composite ink may be spin-coated onto the PCB to make a sensing component. When used for moisture sensing, the sensing component acts as a moisture sensor and responds to the moisture content in the sample (e.g., surrounding environment, air, or breadth) and impacts the conductivity of the sending component. This change in conductivity is the basic principle in detecting the moisture present in the sample. The change in conductivity across the moisture sensor may be measured through the change in voltage across a known resistor connected in series with the sensing component. A constant voltage may be applied to the series connection of the sensing component and the fixed resistor. A continuous voltage change across the fixed resistor may be observed, e.g., for about 10 seconds to about 5 minutes, about 10 seconds to about 1 minute, about 15 seconds to about 45 seconds, or about 30 seconds. The data generated may be sent to any known computation device or processor, such as a microprocessor, to be cleaned and analyzed to output the moisture content in the surrounding. The sensor may be calibrated at the outset of the process with a system of known moisture content and the specific voltage output. The processor may generate a graph showing voltage (mV) and moisture content in ppm.

The carbogenic nanoparticle (CGNP)-conducting polymer composite ink may respond electronically to VOCs. The carbogenic nanoparticle (CGNP)-conducting polymer composite ink may be able to detect very low content of VOC (˜ppb), for example, as low as 10 ppb.

A carbogenic nanoparticle-based conducting polymer composite material synthesized as colloidal suspension in water is disclosed. The composite ink made from the composite material shows selective chemo resistivity by showing specific patterns in current-voltage (I-V) characteristics. The composite ink may be converted into a stable thin film by spin-coating and used in the sensing and detection of moisture and VOCs under average temperature, pressure, and humidity conditions. In an embodiment, the thin film is able to detect the very low content of the analyte (˜ppb), for example, as low as 10 ppb.

Another embodiment is directed to a method of making a R-GO-conducting polymer composite material. Another embodiment is directed to a method of making a R-GO-conducting polymer composite ink. Another embodiment is directed to a method of making a R-GO-PPy composite material. Another embodiment is directed to a method of making a R-GO-PPy composite ink.

An embodiment is directed to a method of making a R-GO-conducting polymer composite ink comprising the steps of: preparing graphene oxide (GO); suspending the GO in water; adding an iron (II) salt and a polymer having either a —COOH or a —SO₃H group to the suspension to make an R-GO suspension; acidifying the R-GO suspension; adding a monomer of the conducting polymer to the acidified R-GO suspension to make a R-GO-conducting polymer composite suspension; evaporating the composite suspension to reduce the volume to the composite ink. The polymer having either a —COOH or a —SO₃H group may be polystyrene sulfonate (PSS), polyacrylic acid, carboxymethyl cellulose, alginate, pectin, polyphenylene sulphonic acid, and any other sulphonated polymer. The polymer having either a —COOH or a —SO₃H group may be PSS. The conducting polymer may be PPy, PANI, PTH, PA, PPP, PPV or PF. The conducting polymer may be PPy. In the step of adding the conducting polymer, the acidified solution may be kept cool, for example, below about 15° C., below about 10° C., from about 5° C. to about 15° C., or from about 5° C. to about 10° C. The suspension may be evaporated until the viscosity of the composite ink is about 20 mPa·s to about 30 mPa·S, about 20 mPa·s to about 26 mPa·S, about 22 mPa·s to about 26 mPa·S, or about 23 mPa·s to about 25 mPa·S within a temperature of about 25° C. to about 50° C.

An embodiment is directed to a method of making a R-GO-conducting polymer composite ink comprising the steps of: synthesizing R-GO; mixing FeCl₂ and a polymer having either a —COOH or a —SO₃H group, such as, but not limited to, polystyrene sulfonate, to initiate synthesis of R-GO; coating R-GO with a conducting polymer, such as PPy, via in situ surface-confined polymerization and electrostatic interactions to make R-GO-conducting polymer composite suspension. Optionally, a further step includes evaporating the suspension to reduce the volume to the composite ink. The resulting R-GO-conducting polymer composite is highly dispersible in water and showed good performance as the active sensor material for moisture and VOC sensor, respectively.

The terms used in connection with these embodiments (methods of making) have the same meanings and definitions as discussed above.

The features and advantages of the present invention are more fully shown by the following examples which are provided for purposes of illustration, and are not to be construed as limiting the invention in any way.

### Examples

For the following examples, the citric acid (99.7%) was purchased from Himedia Chemicals. Poly (sodium 4-styrene sulfonate, 99.8%, M.W. 70,000) (PSS) & Iron (III) chloride hexahydrate were purchased from Sigma-Aldrich and used as received without further purification. Pyrrole (98+%) was purchased from Alfa Aesar and used after distillation. DOWEX® 50 WX2 hydrogen form resin was purchased from Sigma Aldrich. Graphite fine powder 98% was purchased from LOBA CHEMIE. All the solvents used were purchased from Fluka and used without further purification. Snakeskin dialysis tubing (3.5K MWCO, 35 mm dry I.D.) was purchased from Thermo Scientific. Milli-Q water was used in all experiments. The VOCs tested for sensor properties were obtained as commercial products. All the measurements were performed at room temperature (ca. 25° C.) unless otherwise mentioned.

### Example 1: Synthesis of CQD-PPy Ink

### Step 1: Synthesis of Carbon Quantum Dots

Carbon Quantum Dot passivated with poly (sodium 4-styrene sulfonate), i.e., PSS-CQD, were prepared in the earlier reported one-step pyrolysis method. Citric acid was used as a precursor for CQD. Bhattacharjee, L., et al., “Conducting Carbon Quantum Dots—A Nascent Nanomaterial,” J. Mater. Chem. A, 3, pp. 1580-1586 (2015).

### Step 2: Preparation of Protonated Carbon Quantum Dots (PSS-CQDH⁺) by Ion-Exchange Method

About 0.7 grams of CQDs (prepared in preceding step) were suspended in 100 ml of milli-Q water. The as-prepared CQDs were used without purification; the CQD suspension was passed through DOWEX® H ion exchange resin packed in a burette column. Before being used in the column, the ion exchange resin was washed multiple times with Milli-Q water. After passing through the column, the aqueous suspension of CQD was collected at the bottom of the column. The suspension was passed through the column a second time. As a result of this process, the Na⁺ ions of the sulfonate group in the CQD suspension were exchanged with H⁺ ions of the resin. The final pH of the suspension after passing twice through the column was found to be 2.5.

### Step 3: Preparation of CQD-Fe Surface

In a typical preparation of CQD-Fe′, Aqueous FeCl₃, 6H₂O salt solution acidified with concentrated HCl was added to 100 ml protonated CQD solution so that the final concentration of FeCl₃ in the medium was 2 mM. The solution was stirred for 24 hours. The solution was dialyzed for 48 hours against Milli-Q water using snakeskin dialysis tubing to get rid of excess ions present therein. Two other different amounts of Fe(III) ions loaded CQD suspension, which was prepared by varying the iron (III) salt concentrations in the media to 4.5 mM and 3 mM, respectively. It was found that the addition of a high amount, i.e., more than 4.5 Mm, of FeCl₃ to the colloidal CQDs suspension led to partial flocculation of the colloid, and hence a decrease in colloidal stability. The protonated CQDs suspension, which was treated with 2 mM Fe(III) salt solution, was enough to catalytically oxidize pyrrole (Py) on the CQD surface without harming colloidal stability. All the data provided here are based on the CQD suspension with 2 mM FeCl₃ concentration.

### Step 4: Preparation of Polypyrrole (PPy) on CQD-Fe Surface

25 ml of CQD-Fe′ prepared with the 2 mM FeCl₃ solution was taken in a specially designed amber glass vessel. N2 was continuously passed through the solution to maintain an inert atmosphere and remove dissolved oxygen present. The solution was kept below room temperature by about 7° C. through the continuous circulation of chilled water. 10 μl of distilled Py was added to the solution, and the solution was stirred continuously. Under the nitrogen atmosphere and the chilled condition, the Fe′ initiated polymerization, and the solution started becoming black within ten minutes. A kinetic study was done over time. The aliquot was withdrawn from time to time, and the UV-vis spectra of the withdrawn samples were recorded after proper dilution, i.e., the addition of 2 ml of water to a 200 μl sample. The kinetic study revealed that within two hours, the reaction was over. The resulting CQD-PPy solution was highly colloidal.

### Step 5: Preparation of Ink with CQD-PPy

The CQD-PPy suspension was highly stable, i.e., no visible precipitation, even after one month. 10 ml of the suspension was slowly evaporated on a water bath to get a stable ink-like consistency. The final volume (about 25 ml) was concentrated to 2 ml. The viscosity (ii) of CQD-PPy composite ink was 26 mPa·s to 26.5 mPa·s within a temperature range between about 25° C. to about 50° C. Zeta potential of CQD-PPy composite ink was about −10 mV having hydrodynamic radius from about 50 nm to about 120 nm.

### Example 2: Synthesis of R-GO-PPy Composite Ink

### Step 1: Preparation of Graphene Oxide (GO)

Graphene oxide was prepared from fine graphite powder using the established Hummers method. 0.5 g of fine graphite powder was mixed with 0.5 g sodium nitrate, and to the mixture 25 ml conc. sulfuric acid was added. The mixer was kept on an ice bath. The mixer was stirred vigorously for two hours at a temperature of about 0° C. to about 5° C.

After two hours, about 3 g of potassium permanganate was added slowly to the suspension, while the suspension was stirred and kept at a temperature of about 15° C. The ice bath was then removed, and stirring was continued for 12 hours and a temperature of about 35° C. until the suspension became partly brown in color.

The suspension was slowly diluted with the addition of about 50 ml of water. During the addition of water to concentrate the acid, the entire setup was again kept on an ice bath as the temperature was rapidly elevated to about 98° C. An additional 100 ml water was immediately added. The color of the suspension became brown. The solution was stirred for about another two hours.

As a final step, the suspension was treated with 5 ml of hydrogen peroxide to terminate the reaction. The final color of the suspension was brownish-yellow. The GO sample was washed with dil. HCl and water for several times and dried in oven at 90° C.

### Step 2: Preparation of R-GO-PPy Composite in a One-Pot Synthetic Route

0.2 g GO was weighed and dispersed in 20 ml DD water. The suspension was stirred well on a magnetic stirrer. About 0.046 g FeCl₂, 4H₂O was added to the suspension under vigorous stirring, followed by the addition of about 0.036 g of PSS polymer. The whole system stirred for an hour, and about 1 ml of dilute HCl was added drop-wisely to the suspension. The temperature of the suspension was kept below 10° C., and 10 μl distilled pyrrole was added to the suspension while it was stirred. Within 30 seconds of the addition, the entire suspension turned black, confirming the formation of PPy in the medium.

The black suspension was evaporated to reduce the volume to get the required ink-like consistency. The viscosity (ii) of R-GO-PPy composite ink was 23.5 mPa·s to 24.6 mPa·s within a temperature range between about 25° C. to about 50° C. Zeta potential of R-GO-PPy composite ink was about −4.38 mV having hydrodynamic radius from 1100 nm to 1700 nm with signs of aggregations.

### Example 3: X-Ray Photoelectron Spectroscopy (XPS)

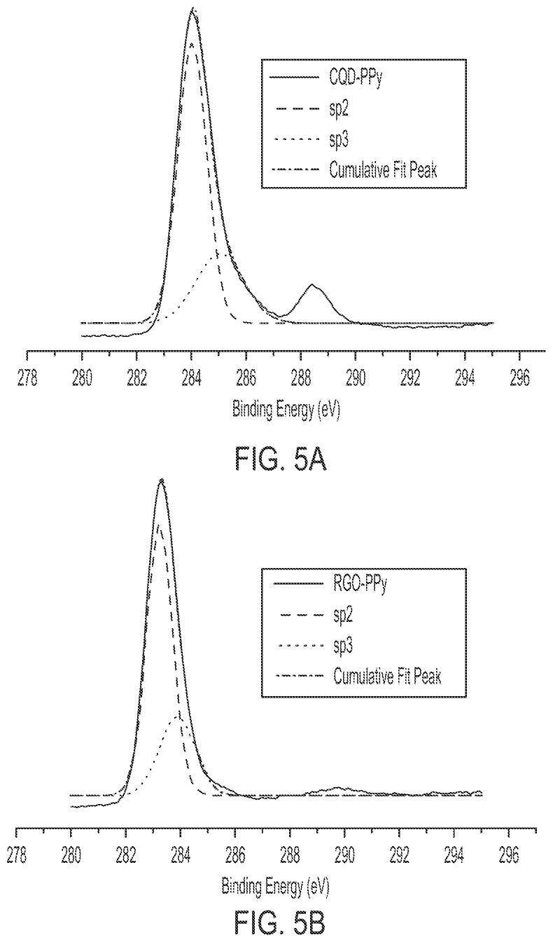

A sample of the CQD-PPy composite ink and a sample of the R-GO-PPy composite ink were tested by x-ray photoelectron spectroscopy. For each experiment, a sample of the composite ink was drop coated on a glass slide and the sample was dried before analyzing in XPS in accordance with known processes and standard techniques. FIGS. 5A and 5B are the graphical results showing C1s core level XPES spectra of CQD-PPy composite ink and RGO-PPy composite ink, respectively.

The results for CQD-PPy composite ink showed: sp2 carbon concentration 71% and sp3 carbon concentration 29%. For CQD-PPy composite ink, the graph shows a peak for sp2 hybridization around 284 eV; a corresponding peak for sp3 hybridization comes at around 285 eV accompanied by some higher energy peaks due to presence of oxygen linkages.

The % sp2 character of the CQD-PPy composite ink was unexpected. Literature suggests R-GO sp2 character as 70.5%. Here, it was found that CQD-PPy composite ink sp2 is 71%. This shows that the CQD matches with known carbon nanomaterials. This was unexpected because CQD-PPy composite ink was prepared from simple citric acid where as preparation of R-GO-PPy composite ink was made from well defined graphitic carbon material. This is important and newly found and means that the CQD-PPy composite inks will have better conducting properties due to high sp2 character compared to other CQDs prepared in other literature reports. Thus better chemiresistive sensors can be designed from the CQD-PPy composite ink disclosed herein.

The results for R-GO-PPy showed: sp2 carbon concentration 71.7% and sp3 carbon concentration 28.3%. The spectrum of RGO-PPY shows an intense peak at 283.5 eV attributed to carbon with sp2 hybridization. The peak for sp3 hybridization (284 eV) accompanied by some shoulders at higher binding energies due to presence of oxygen linkage.

### Example 4: Preparation of a Moisture Sensor

R-GO-PPy composite ink was prepared using the above processes and spin-coated onto a PCB. For spin-coating, a single-sided copper cladded FR4-PCB was pre-treated under a UV lamp (360 nm) for 30 min to make the surface hydrophilic in order to have better adhesion of the ink on the metal and Bakelite surface of the PCB. R-GO-PPy composite ink was coated as a thin film on the PCB according to the following steps:

Step 1: The substrate PCB was placed horizontally on a rotating disk of a spin coater (EZ-spin A1, Apex Instrument) and vacuum was applied;

Step 2: The substrate was covered with 0.5 ml of the R-GO-PPy composite ink;

Step 3: The PCB was rotated for 30 s at a speed 1500 RPM;

Step 4: The PCB was rotated for 30 s at a speed 2500 RPM; and

Step 5: The PCB was rotated for 30 s at a speed 5000 RPM.

Steps 2-5 were repeated to obtain double layer of the R-GO-PPy composite ink of about 20 nm in thickness.

The coated PCB was kept under vacuum for 2 hours before use in a sensor device. R-GO-PPy composite ink was spin-coated onto the PCB to function as a moisture sensor and respond to the moisture content in the surroundings and impacts the sensor's conductivity. The change in conductivity across the moisture sensor was measured through the change in voltage across a known resistor connected in series with the senor. A constant voltage was applied to the series connection of the sensor and the fixed resistor. A continuous voltage change across the fixed resistor was noted for 30 seconds. The data generated was then sent to a microprocessor to be cleaned and analyzed to output the moisture content in the surrounding. The sensor was first calibrated with a known system of moisture content and the specific voltage output. A linear graph fitting was done to produce a database for the voltage and moisture content in ppm level for the sensor to detect the moisture content accurately.

FIGS. 4A and 4B show the response of CQD-PPy composite ink and R-GO-PPy composite ink, respectively, when exposed to humidity. These graphs confirm reversible chemi-resistance of CQD-PPy composite ink and R-GO-PPy composite ink as sensing components in a moisture sensor. “in-time” is when the probe was inserted and “out time” is when the probe was withdrawn from the sample. No heat was applied. As shown in the figures, there is a period of latency of the sensing component between when the probe is inserted and system detects moisture. As shown in the figures, 2 minutes and 3 minutes, respectively, is needed to reach the full voltage. These figures show both the response time needed for detection as well as the time for regeneration (i.e., when the voltage returns to the baseline reading). The sensitivity of these composite inks is good and it was found that the sensor device dries within 50-100 seconds at room temperature and normal conditions. The differences in response values are due to the very high conductivity of R-GO versus CQDs. All adsorption and desorptions were tested at room temperatures

### Example 5: Preparation of a VOC Sensor

CQD-PPy composite ink was prepared using the above processes and spin-coated onto a PCB. For spin-coating, the PCB was pre-treated under a UV lamp (360 nm) for 30 min to make the surface hydrophilic in order to have better adhesion of the ink on the metal and Bakelite surface of the PCB. CQD-PPy composite ink was coated as a thin film on the PCB according to the following steps:

Step 1: The substrate PCB was placed horizontally on a rotating disk of a spin coater and vacuum was applied;

Step 2: The substrate was covered with 0.5 ml of the CQD-PPy composite ink;

Step 3: The PCB was rotated for 30 s at a speed 1500 RPM;

Step 4: The PCB was rotated for 30 s at a speed 2500 RPM; and

Step 5: The PCB was rotated for 30 s at a speed 5000 RPM.

Steps 2-5 were repeated to obtain double layer of the CQD-PPy composite ink of about 30 nm in thickness.

The coated PCB was kept under vacuum for 2 hours before use in a sensor device.

A VOC sensor was built with spin-coating the CQD-PPy composite ink onto the PCB. I-V characteristic curves were generated using the same technique, and specific I-V characteristics curves were measured for the particular VOC. A specific VOC was identified by measuring the voltage and current passing through the fixed resistor. The data was processed in the micro-controller with the pre-identified I-V characteristics of the VOCs. The ink after casting on the device is very stable and scratch resistant as observed. It does not peel off, disperse or dissolve during tests carried out by inserting into the polymer pellets.

### Example 6: Moisture Sensor

Moisture sensing experiments un-edited involving both the inks and a comparative data to show the better performing ink as moisture sensor.

We have tested the sensor in two methods:

Method 1: We have inserted the probe directly inside polymer

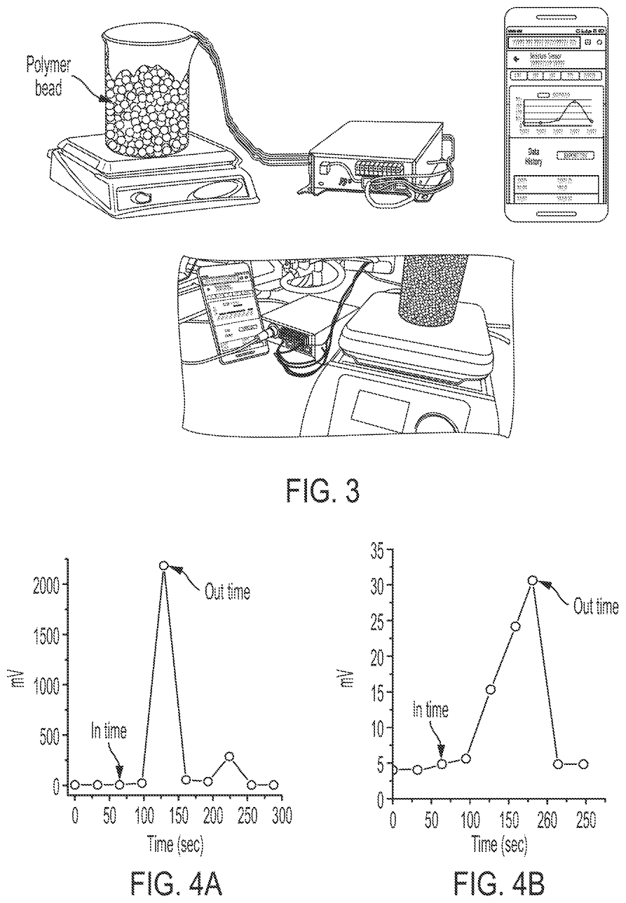

Method 2: We have inserted the probe inside the moisture environment created by heating the sample in a closed vessel (as shown in FIG. 3)

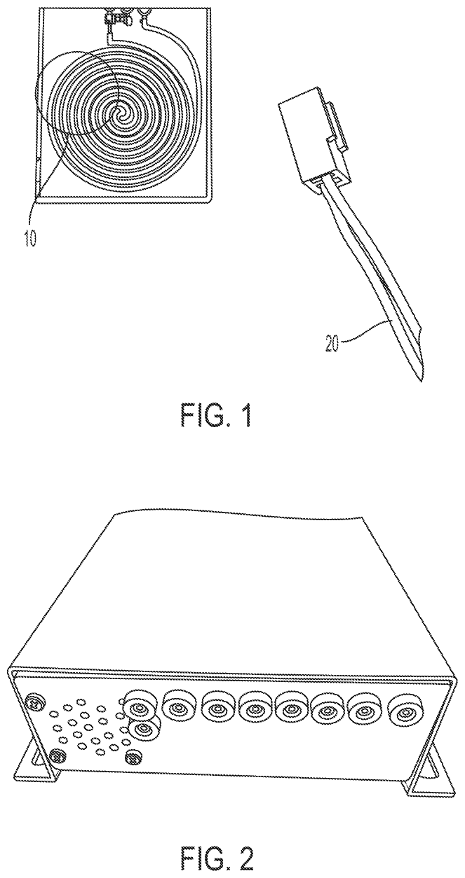

The circular probe, shown in FIG. 1, was found to work well with few limitations. The circular probe tip was designed to be inserted into a sample. The circular probe tip as shown in FIG. 1 is easily detached and can be dried quickly and easily for reuse. The size of the plug may need to be adjusted so that the ink can be applied easily by spin-coating.

The gap and/or the capacitance value in between the electrodes need to be optimized for better sensing. For highly conducting ink, the signal is getting saturated fast. The gap between the electrodes or the number of turns can be manipulated to reduce this effect.

The circular probe 10 may be attached to the detachable wire 20 shown in FIG. 1, which at the other end thereof (not shown) is connected to the Gateway box, which is 110-230 AV supply operated.

The results of running an experiment using the circular probe shown in FIG. 1 indicates that for moisture sensor, the R-GO-PPY composite ink performs well in low moisture region (15 to 100 ppm) compared to that of CQD composite inks.

This data was collected by inserting the device inside the vessel with an open mouth. Hence it took some time before a saturation vapor pressure could be attained and which is a mandatory for quick detection of moisture.

### Example 7: Ink Efficiency

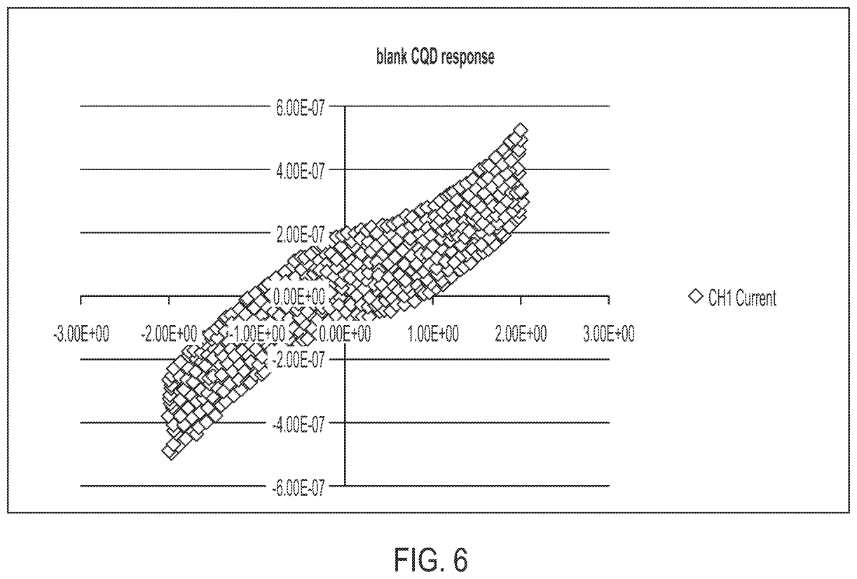

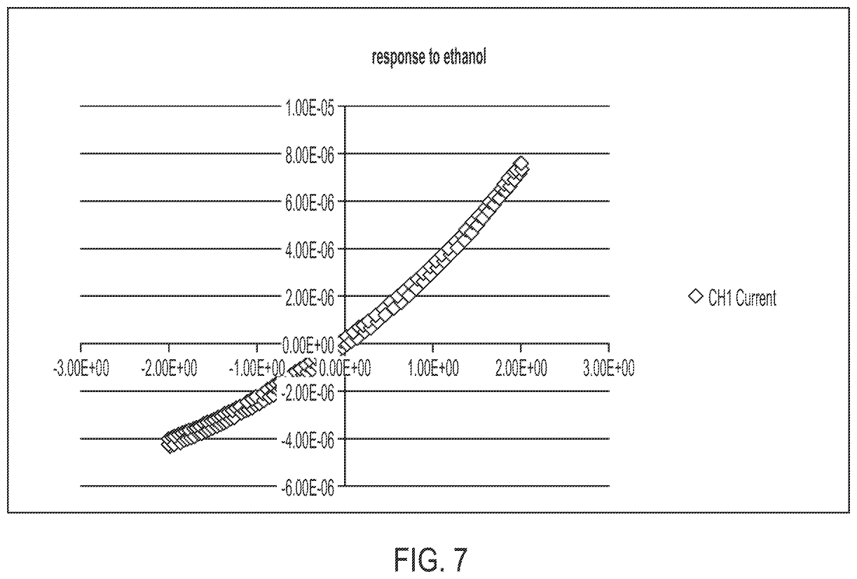

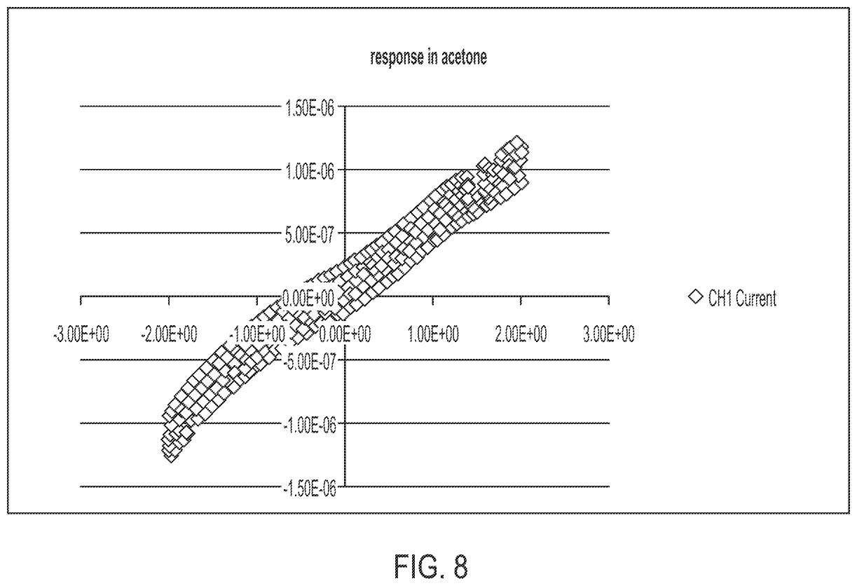

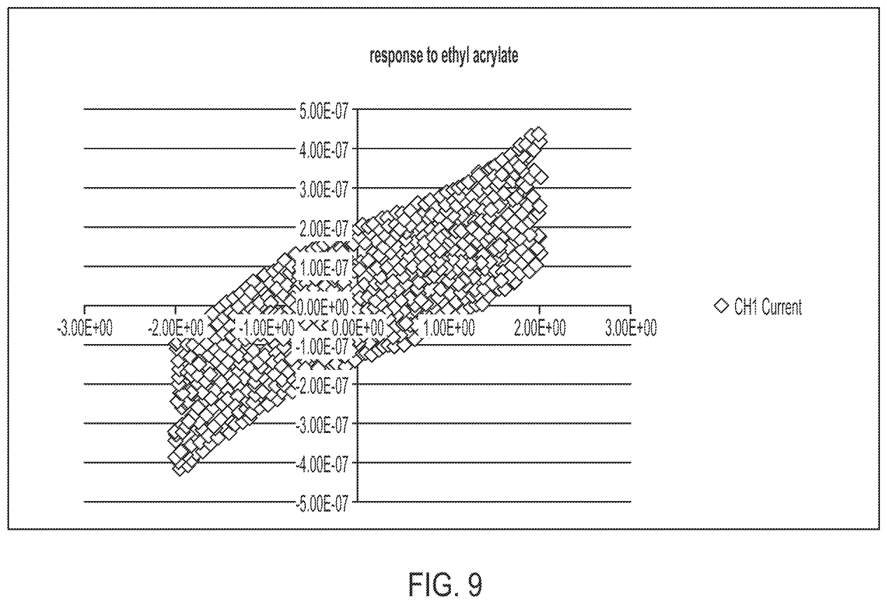

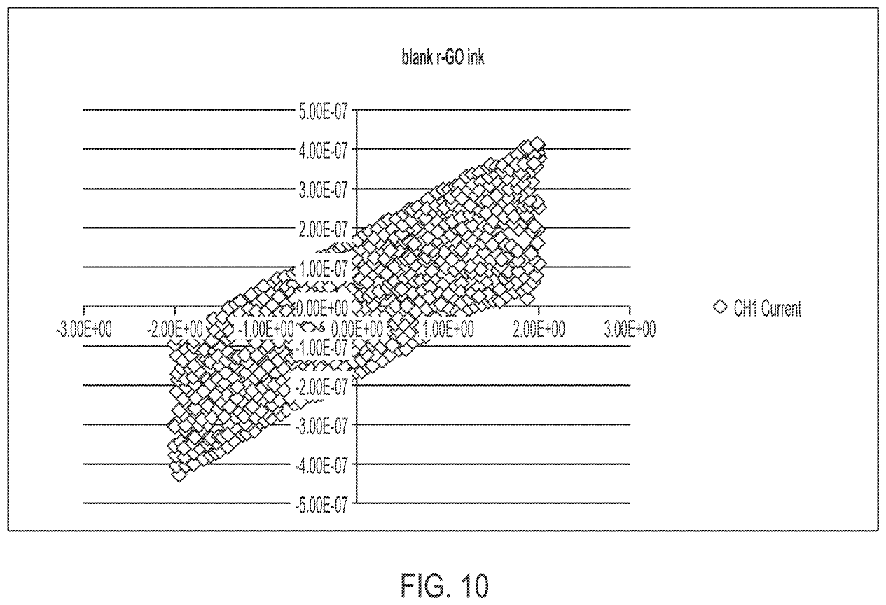

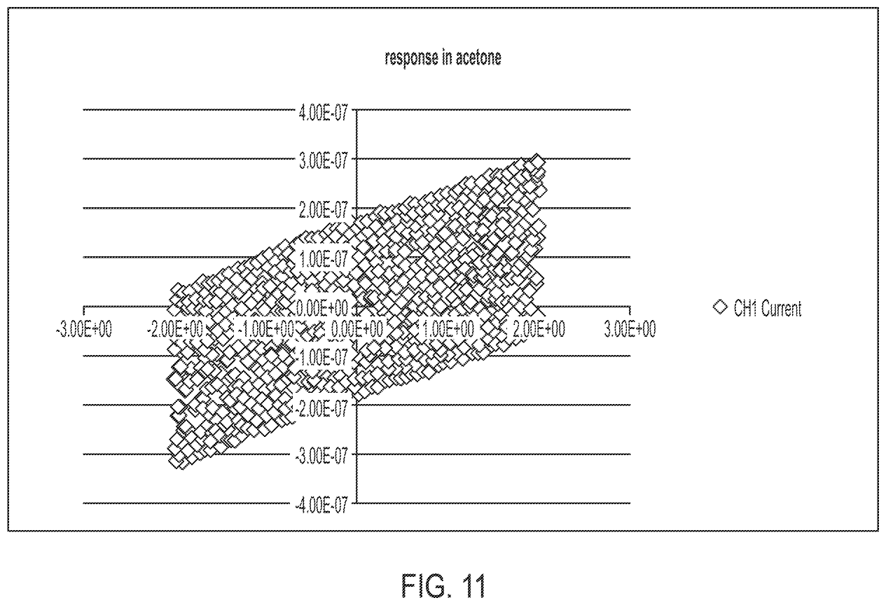

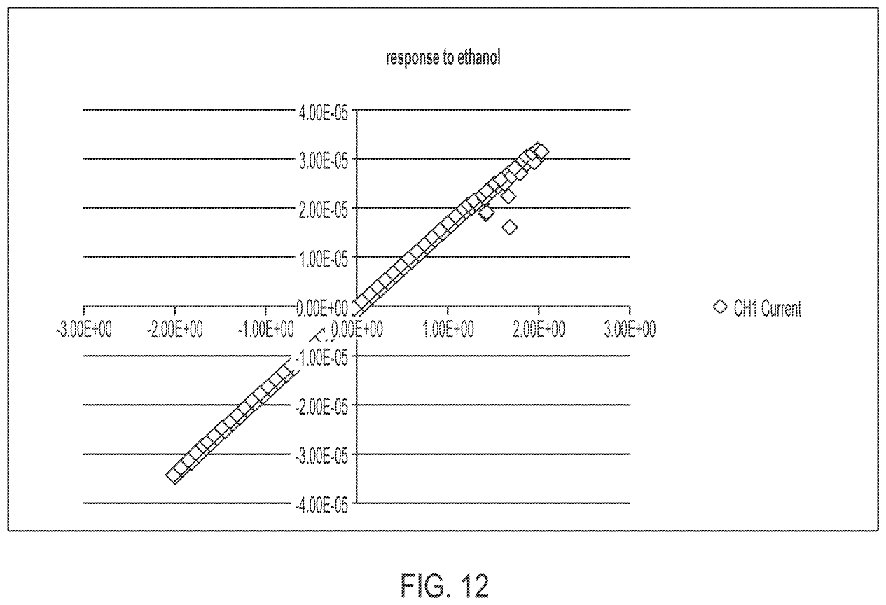

The present experiments were conducted by inserting the probe within a closed VOC saturated chamber. Unlike with an open container in the preceding experiment, in this experiment, there was a recorded response time of 30-50 sec collected for two different devices. The first sensor device incorporated CQD-PPy composite ink (prepared as set forth above) and the second sensor device incorporated R-GO-PPy composite ink. FIGS. 6-9 show VOC sensing data for a sensor made from PCB coated with CQD-PPy composite ink as processed in the experiment above. FIGS. 10-12 show VOC sensing data for a sensor made from PCB coated with R-GO-PPy composite ink as processed in the experiment above. Desired amount of VOC (ethanol/acetone in ppb level) was injected into a 250 ml closed chamber where sensors were already inserted through an orifice. The different inks respond in similar ways to ethanol but not in the same manner when exposed to acetone. We can conclude from initial findings that based on the dipole moments of the organic solvents of VOCs, the responses of the composite ink responses will be different.

While there have been described what are presently believed to be various aspects and certain desirable embodiments of the disclosure, those skilled in the art will recognize that changes and modifications may be made thereto without departing from the spirit of the disclosure, and it is intended to include all such changes and modifications as fall within the true scope of the disclosure.

What is claimed:

1. A carbogenic nanoparticle-conducting polymer composite ink dispersed in water, wherein the carbogenic nanoparticle is reduced graphene oxide.

2. The composite ink of claim 1, wherein the carbogenic nanoparticle-conducting polymer composite ink is selected from the group consisting of: R-GO-PPy composite ink, R-GO-PANI composite ink, R-GO-PTH composite ink, R-GO-PA composite ink, R-GO-PPP composite ink, R-GO-PPV composite ink, R-GO-PF composite ink, or a combination thereof.

3. The composite ink of claim 1, wherein the carbogenic nanoparticle-conducting polymer composite ink is R-GO-PPy composite ink.

4. The composite ink of claim 1, wherein R-GO-PPy composite ink has a viscosity of about 20 mPa·s to about 30 mPa·s within a temperature range between about 25° C. to about 50° C.

5. A carbogenic nanoparticle-conducting polymer composite ink dispersed in water selected from the group consisting of: R-GO-PPy composite ink, CQD-PANI composite ink, R-GO-PANI composite ink, CQD-PTH composite ink, R-GO-PTH composite ink, CQD-PA composite ink, R-GO-PA composite ink, CQD-PPP composite ink, R-GO-PPP composite ink, CQD-PPV composite ink, R-GO-PPV composite ink, CQD-PF composite ink, R-GO-PF composite ink, or a combination thereof.

6. The composite ink of claim 5, wherein the composite ink has a viscosity of about 20 mPa·s to about 0.15 Pa·s within a temperature range between about 25° C. to about 50° C.

7. The composite ink of claim 5, wherein the composite ink has a zeta potential of about +40 mV to about −40 mV and a hydrodynamic radius from about 40 to about 2000 nm.

8. A thin film coated PCB comprising: a thin film of a carbogenic nanoparticle-conducting polymer composite ink selected from the group consisting of: CQD-PPy composite ink, R-GO-PPy composite ink, CQD-PANI composite ink, R-GO-PANI composite ink, CQD-PTH composite ink, R-GO-PTH composite ink, CQD-PA composite ink, R-GO-PA composite ink, CQD-PPP composite ink, R-GO-PPP composite ink, CQD-PPV composite ink, R-GO-PPV composite ink, CQD-PF composite ink, R-GO-PF composite ink, or a combination thereof; and the PCB.

9. The thin film coated PCB of claim 8, wherein the thin film of the carbogenic nanoparticle-conducting polymer composite ink has a thickness of about 10 nm to about 50 nm.

10. The thin film coated PCB of claim 8, wherein the thin film of the carbogenic nanoparticle-conducting polymer composite ink has a thickness of about 15 nm to about 40 nm.

11. A method of making a thin film coated PCB comprising the steps of:
treating the PCB under a UV lamp at about 260 nm to about 400 nm for about 15 minutes to about 60 minutes; and
spin-coating the treated PCB with a carbogenic nanoparticle-conducting polymer composite ink comprising the steps of:
i) horizontally positioning the treated PCB on a rotating disk of a spin-coating machine, under vacuum;
ii) coating the substrate with a small amount of the composite ink;
iii) rotating the coated PCB at one or more different speeds to obtain a first layer of composite ink on the PCB; and
iv) optionally repeating steps ii and iii one to four times to obtain a double, triple, quadruple or quintuple layer of composite ink to make the thin film on the PCB.

12. The method of claim 11, wherein in step iv), steps ii and iii are repeated one to three times to obtain a double, triple, or quadruple layer of composite ink to make the thin film coat on the PCB.

13. The method of claim 11, further comprising a step v) maintaining the coated PCB under vacuum for at least about 2 hours.

14. The method of claim 11, wherein the step of rotating the PCB at one or more different speeds includes three steps with each step being at a different rotation speed for about 20 seconds to about 60 seconds.

15. The method of claim 11, wherein the step of rotating the PCB at one or more different speeds includes: (a) rotating the coated PCB for about 20 seconds to about 60 seconds at a speed of about 1000 RMP to about 1700 RPM, followed by (b) rotating the coated PCB for about 20 seconds to about 60 seconds at a speed of about 2000 RMP to about 3000 RPM, followed by (c) rotating the coated PCB for about 20 seconds to about 60 seconds at a speed of about 4000 RMP to about 6000 RPM.

16. The method of claim 11, wherein the small amount of composite ink is about 0.1 ml to about 1 ml.

17. The method of claim 11, wherein the thin film of composite ink has a thickness of about 15 nm to about 40 nm.

18. The method of claim 11, wherein the composite ink is selected from the group consisting of: CQD-PPy composite ink, R-GO-PPy composite ink, CQD-PANI composite ink, R-GO-PANI composite ink, CQD-PTH composite ink, R-GO-PTH composite ink, CQD-PA composite ink, R-GO-PA composite ink, CQD-PPP composite ink, R-GO-PPP composite ink, CQD-PPV composite ink, R-GO-PPV composite ink, CQD-PF composite ink, R-GO-PF composite ink, or a combination thereof.

19. The method of claim 11, wherein the composite ink is CQD-PPy composite ink or R-GO-PPy composite ink.

20. The method of claim 19, wherein the composite ink is CQD-PPy composite ink having a viscosity of about 20 mPa·s to about 30 mPa·s within a temperature range between about 25° C. to about 50° C.

21. The method of claim 20, wherein the CQD-PPy composite ink has a zeta potential of about −8 mV to about −12 mV and a hydrodynamic radius from about 40 nm to about 150 nm.

22. The method of claim 19, wherein the composite ink is R-GO-PPy composite ink having a viscosity of about 20 mPa·s to about 30 mPa·s within a temperature range between about 25° C. to about 50° C.

23. The method if claim 22, wherein the R-GO-PPy composite ink has a zeta potential of about −2 mV to about −8 mV and a hydrodynamic radius from about 900 to about 2000 nm.

24. A VOC sensor comprising the thin film coated PCB of claim 8.

25. The VOC sensor of claim 24, wherein as low as about 10 ppbs of VOCs are detected by the sensor.

26. A moisture sensor comprising the thin film coated PCB of claim 8.

27. A method of detecting moisture, VOCs or both in a sample using the thin film coated PCB of claim 8.

28. A method of making a R-GO-conducting polymer composite ink comprising the steps of:
a. Preparing graphene oxide (GO);
b. Suspending the GO in water;
c. Adding an iron (II) salt and a polymer having either a —COOH or a —SO₃H group to the suspension to make an R-GO suspension;
d. Acidifying the R-GO suspension;
e. Adding a monomer of the conducting polymer to the acidified R-GO suspension to make a R-GO-conducting polymer composite suspension;
f. Evaporating the composite suspension to reduce the volume to the composite ink.

29. The method of claim 28, wherein the polymer having either a —COOH or a —SO₃H group is polystyrene sulfonate (PSS), polyacrylic acid, carboxymethyl cellulose, alginate, pectin, polyphenylene sulphonic acid, or other sulphonated polymer.

30. The method of claim 29, wherein the polymer having either a —COOH or a —SO₃H group is PSS.

31. The method of claim 30, wherein the conducting polymer is PPy, PANI, PTH, PA, PPP, PPV or PF.

32. The method of claim 31, wherein the conducting polymer is PPy.
